(19) United States
(12) Patent Application Publication
(10) Pub. No.: US 2015/0232823 A1
(43) Pub. Date: Aug. 20, 2015

(54) PROCESS FOR PURIFYING GROWTH FACTORS FROM MILK AND PRODUCTS THEREOF

(71) Applicant: Murray Goulburn Co-Operative Co. Ltd., Southbank, Victoria (AU)

(72) Inventor: Andrew Brown, Point Cook (AU)

(21) Appl. No.: 14/434,272

(22) PCT Filed: Oct. 8, 2013
(86) PCT No.: PCT/AU2013/001154

§ 371 Date: Apr. 8, 2015

(30) Foreign Application Priority Data
Oct. 8, 2012 (AU) 2012904391
Apr. 12, 2013 (AU) 2013204850

(51) Int. Cl. C12N 9/22; C07K 1/34; C07K 14/79; C07K 14/475

(52) U.S. Cl. CPC C12N 9/22; C07K 14/475; C07K 1/34; C07K 14/79; A61K 38/00

(57) ABSTRACT

The invention provides process for purifying RNAses and growth factors from milk or lactoferrin, the process comprising subjecting the milk or lactoferrin to filtration to separate it into a retentate fraction comprising lactoferrin and a permeate fraction comprising growth factors and/or RNAses, wherein prior to and/or during filtration the milk or lactoferrin is subjected to salt treatment such that growth factors and/or RNAses flow into the permeate. The invention also provides RNAses and growth factors obtained from the process of the invention.

### FIELD

This invention relates generally to processes for purifying proteins of interest from milk.

### BACKGROUND

Milk from domestic animals has been used as a source of proteins and other products for the food and pharmaceutical industries for many years, and a variety of techniques are known for isolating these products. Milk is a colloidal suspension composed primarily of fats, lactose and proteins in water. Among ruminants and laboratory animals, milk contains an average of 30 to 140 grams of protein per litre, or about 4-17% by weight, depending on the species. The bulk of these proteins are caseins, which are complexed with calcium and phosphate in supramolecular structures known as micelles. The other major class of milk proteins is whey proteins, predominantly comprised of beta-lactoglobulin and alpha-lactalbumin, but also including lactoferrin, immunoglobulins, and serum albumin.

Milk proteins usually are isolated by a combination of processes including membrane filtration techniques as well as ion exchange adsorption procedures.

Lactoferrin is an 80 kD iron-binding glycoprotein found naturally in biological fluids such as saliva, bile, bronchial mucus, gastrointestinal fluids, cervico-vaginal mucus, seminal fluid, and milk. The richest source of lactoferrin is mammalian milk and colostrum. The concentration of lactoferrin in bovine skimmed milk is usually small, typically between 80-200 mg/ml depending on factors including the pasteurisation and other pre-treatment history of the skimmed milk. After precipitation of the casein present in milk the concentration of lactoferrin in bovine whey is typically 10-100 mg/ml depending on the physical and chemical pre-treatment of the whey.

Growth factors are present in milk to various degrees, particularly in lactoferrin containing fractions. Bovine milk contains RNAses such as RNAse 5 and growth factors including IGF-1, IGF-2, PDGF, FGF-basic, EGF, FGF-acidic and VEGF. Such growth factors each have a molecular weight of 30 kD or less (RNAse 5 has a molecular weight of 17 kD). Milk fractions enriched for growth factors and RNAses are prepared in the art using ion exchange chromatography (for example as described in International Patent applications PCT/AU91/00303 and PCT/AU2007/001719).

Milk fractions enriched for RNAse 5 and growth factors have multiple postulated biological roles including promoting muscle growth, neuroprotection, promoting osteogenesis and treating neurological diseases or disorders, spinal injuries or diseases, bone diseases or disorders, diseases involving glucose homeostasis, wound healing, or for providing nervous system functional support and managing metabolic diseases,

It is an aim of a preferred embodiment of the present invention to provide an improved method for purifying growth factors and RNAses from milk, particularly bovine milk, to improve purity.

All references, including any patents or patent applications, cited in this specification are hereby incorporated by reference. It will be clearly understood that, although a number of prior art publications are referred to herein, this reference does not constitute an admission that any of these documents forms part of the common general knowledge in the art.

### SUMMARY

A first aspect provides a process for purifying RNAses and growth factors from milk or lactoferrin, the process comprising subjecting the milk or lactoferrin to filtration to separate it into a retentate fraction comprising lactoferrin and a permeate fraction comprising growth factors and/or RNAses, wherein prior to and/or during filtration the milk is subjected to salt treatment such that growth factors and/or RNAses flow into the permeate.

When purifying lactoferrin from milk using membrane filtration with a 30 kD or 50 kD cut off the inventors found that the lactoferrin retentate was contaminated with growth factors and RNAses. As these growth factors and RNAses have a molecular weight of less than 30 kD they should have passed through the membrane and into the permeate and not be present as an impurity in the retentate lactoferrin fraction. Accordingly the discovery of growth factors and RNAses in the retentate was surprising. The inventors found that the addition of a large amount of salt to the milk prior to filtration removed the growth factors and RNAses from the retentate and concentrated them in the permeate.

Without wishing to be bound by theory the inventors propose that under normal conditions the RNAses and growth factors in milk aggregate or otherwise form a mass that is larger than their individual molecular weights. It is proposed that the salt treatment causes the RNAses and growth factors to disaggregate or disassociate.

In an alternative form the process of the first aspect provides a process for purifying RNAses and growth factors from milk or lactoferrin, the process comprising subjecting the milk or lactoferrin to filtration to separate it into a retentate fraction comprising lactoferrin and a permeate fraction comprising growth factors and/or RNAses, wherein prior to and/or during filtration the milk or lactoferrin is subjected to salt treatment capable of disaggregating or disassociating any mass of RNAses or growth factors such that growth factors and/or RNAses flow into the permeate.

Without wishing to be bound by theory the inventors alternatively propose that under conditions of low ionic strength, protein aggregates may become associated with the membrane, thereby forming a layer with a smaller apparent pore size to that of the membrane, which prevents RNAses and growth factors in milk passing through the membrane. It is proposed that the salt treatment prevents formation of or removes the protein layer, allowing any RNAses and growth factors to pass through the membrane into the permeate.

Accordingly in an alternative form the process of the first aspect provides a process for for purifying RNAses and growth factors from milk or alctoferrin, the process comprising subjecting the milk or lactoferrin to membrane filtration to separate it into a retentate fraction comprising lactoferrin and a permeate fraction comprising growth factors and/or RNAses, wherein prior to and/or during filtration the milk or lactoferrin is subjected to salt treatment capable of separating any RNAses or growth factors from the membrane such that growth factors and/or RNAses flow into the permeate.

A second aspect provides RNAses and growth factors obtained from the process of the first aspect.

In one embodiment the RNAses and growth factors of the second aspect are subjected to further purification.

A third aspect provides use of RNAses and growth factors of the second aspect for the treatment of diseases caused by viruses, bacteria, or fungi and their toxins, to target pathogens which cause infections of human mucosal surfaces, to promote angiogenesis, for treating a disorder characterised by elevated myostatin, for treating disorders where the interaction between follistatin and angiogenin can be used to improve function in tissues, for promoting muscle growth, for improving recovery of muscle from injury or use, for improving muscle strength, for improving exercise tolerance, for increasing the proportion of muscle, for decreasing fat, for decreasing an individual's fat to muscle ratio, for treating neurological diseases or disorders, for treating spinal injuries or diseases, for treating bone diseases or disorders, for treating diseases involving glucose homeostasis, for wound healing, or for providing neuroprotection, nervous system functional support, managing metabolic diseases and/or increasing the bone density of an individual, for treating inflammation, to treat cancer, to treat cancer cachexia, to treat periodontitis and in all other uses of growth factors and RNAses known to persons skilled in the art.

In an alternative form the third aspect provides RNAses and growth factors obtained from the process of the first aspect for use as proposed in the third aspect or for use in the manufacture of a medicament for use as proposed in the third aspect.

### BRIEF DESCRIPTION OF FIGURES

FIG. 1 shows ultrafiltration of a mixture of cationic milk proteins through a 50 kDa membrane, in the presence of sodium chloride (100 mS), selectively allows the transmission of RNAses and other growth factors.

FIG. 2 shows ultrafiltration of a mixture of cationic milk proteins through a 50 kDa membrane, in the presence of sodium chloride (20 or 40 mS), selectively allows the transmission of RNAses and other growth factors, which do not permeate in the absence of sodium chloride (0 mS).

FIG. 3 shows increased transmission of growth factors, such as the RNAses. Growth factor transmission increases quickly between 0 mS and 20 mS.

### DETAILED DESCRIPTION

The present invention provides improved methods for purifying RNAses and growth factors from milk or lactoferrin or enriching milk or lactoferrin for such products.

The inventors have recognised the need for a process which allows the preparation of enriched RNAse and growth factor fractions in an efficient manner.

The terms “purified” or “enriched” as used herein in relation to RNAses or growth factors means that the RNAse/growth factor:total protein ratio present in the permeate is increased relative to the ratio present in the milk or lactoferrin before the filtration step. For the fraction to be considered enriched or purified, it should have an RNAse/growth factor content of at least 1, 2, 3, 4, 5, 6, 7, 8, 9, 10, 11, 12, 13, 14, 15, 16, 17, 18, 19, 20, 22, 25, 27, 28, or 30, 40, 50, 60, 70, 80, 90, 95, 96, 97, 98, 98, or 99% w/w higher than in milk or lactoferrin before the filtration step.

The process of the first aspect seeks to increase the purity of the RNAses and growth factors in the permeate.

As used herein, the term “fraction” refers to a partially purified portion of milk, particularly a fraction comprising lactoferrin.

Use of the term “efficient” is taken to mean an inexpensive and quick process when compared to methods which are currently employed to enrich for proteins.

Reference herein to milk includes whole milk, skim milk, buttermilk, whey (such as acid or cheese/renneted whey) or a whey derivative (such as whey protein concentrate or whey protein isolate flow through), and colostrum. It also includes milk fractions, for example fractions that have been subjected to purification steps such as cation exchange chromatography. Such fractions include milk basic protein and fractions containing lactoferrin.

It will be apparent to those skilled in the art that the milk may be obtained from any lactating animal, e.g. ruminants such as cows, sheep, buffalos, goats, and deer, non-ruminants including primates such as a human, and monogastrics such as pigs. It is preferred that skim milk which is derived from whole cow's milk is used in the process of the present invention. The filtration used in the process of the first aspect comprises membrane filtration. In one embodiment the membrane has a size cut off of 25 kD, 30 kD, 35 kD, 40 kD, 45 kD, 50 kD, 55 kD, 60 kD, 65 kD, 70 kD or 75 kD. The cut off is preferably 30 kD or over and 50 kD or less.

The filtration may involve ultrafiltration or diafiltration or both.

The salt treatment used in the process of the first aspect involves adding sufficient salt so that the ionic strength of the milk or lactoferrin is at least 0.2 M (1.1%) NaCl or equivalent. In one embodiment the ionic strength is maintained at least this level for a period required to obtain the required increase in concentration of RNases and/or growth factors in the permeate. This period may be at least 10 minutes, 15 minutes, 20 minutes, 30 minutes, 45 minutes, 1 hour, 2 hours, 3 hours, 4 hours, 5 hours, 6 hours, 7 hours, 8 hours, 9 hours, 10 hours, 11 hours, 12 hours or more depending on the feed material and the increase in concentration or purity desired.

In an alternative the salt treatment used in the methods of the first aspect involves adding sufficient salt so that the conductivity of the milk or lactoferrin is at least 20 mS. In one embodiment the conductivity is maintained at at least this level during the entire filtration step.

Generally the milk or lactoferrin will have an ionic strength of substantially less than 0.2M NaCl or equivalent or less that 20 mS conductivity and hence the salt treatment involves the addition of salt to increase the ionic strength or conductivity of the milk or lactoferrin to the desired level. However in some circumstances, for example when the milk is a fraction that has been subjected to cation exchange, the milk will have an ionic strength that may be at least 0.2 M NaCl or equivalent or the conductivity will be 20 mS or more. Generally fractions from cation exchange are subjected to treatment to remove salt (e.g. diafiltration with water). However according to the process of the first aspect the salt treatment is such to maintain the ionic strength of the milk at at least 0.2 M (1.1%) NaCl or equivalent during the filtration step or to maintain the conductivity of the milk at at least 20 mS during the filtration step.

As used herein “conductivity” is the ability of a material to conduct electric current. Conductivity is generally measured using a conductivity meter for example a Hach Sension 5. Persons skilled in the art would be aware of suitable alternative means to measure conductivity. There is a generally linear relationship between sodium ion concentration and conductivity.

The salt used in the salt treatment is not limited and alternatives to NaCl would be known to the person skilled in the art. For example any soluble, non-toxic buffer can be used such as the soluble sodium, potassium, calcium, magnesium or lithium salts of chloride, citrate, phosphate, acetate, sulphate, bicarbonate, hydroxide, imidazole, or maleate. Synthetic zwitterion buffers such as Trizma, HEPES or tricine may also be used.

To allow separation of the RNAses and growth factors from lactoferrin the ionic strength of the milk or lactoferrin must be at least 0.2 M (1.1%) NaCl or equivalent or more prior to the filtration step. In one embodiment the ionic strength of the milk is increased by adding 1.1%, salt, 1.5% salt, 2% salt, 2.5% salt, 3% salt, 3.5% salt, 4% salt, 4.5% salt, 5% salt, 5.5% salt, 6% salt or more. In another embodiment the ionic strength of the milk or lactoferrin is increased to 0.2M, 0.22M, 0.24M, 0.26M, 0.28M, 0.30M, 0.32M, 0.34M, 0.36M, 0.38M, 0.40M, 0.42M, 0.44M, 0.46M, 0.48M, 0.50M, 0.6M, 0.7M, 0.8M, 0.9M, 1.0M NaCl or equivalent or more. In another embodiment, the conductivity is 20 mS, 30 mS, 40 mS, 50 mS, 60 mS, 70 mS. 80 mS, 90 mS, 100 mS, 110 mS, 120 mS or more.

In a preferred embodiment the salt treatment involves the addition of 0.2-0.5M NaCl or KCl to the milk or lactoferrin prior to the filtration step.

In one embodiment the salt treatment is carried out at 4-10 degrees.

In one embodiment the salt treatment is carried out at 10-30 degrees.

In one embodiment the salt treatment is carried out at 30-50 degrees.

In one embodiment the salt treatment is carried out at 50-70 degrees.

In one embodiment the salt treatment is carried out at less than 20 degrees.

In another embodiment the salt treatment is carried out at 50 degrees or higher as at temperatures in excess of 50 degrees coliform formation is reduced. However as lactoferrin denatures at roughly 70 degrees performing the salt treatment at 60 degrees or above may result in decreased yield.

In one embodiment the filtration step is carried out at 4-10 degrees.

In one embodiment the filtration step is carried out at 10-30 degrees.

In one embodiment the filtration step is carried out at 30-50 degrees.

In one embodiment the filtration step is carried out at 50-70 degrees.

In one embodiment the salt treatment is carried out at atmospheric pressure.

In one embodiment the filtration step is carried out at a transmembrane pressure less than 2.5 Bar per membrane, more likely less than 2.0 Bar, more likely again less than 1.5 Bar but ideally 1.0-1.4 Bar, although a pressure of 0.0-1.0 Bar could also work acceptably but have a lower transmembrane flux.

“Treating” or “treatment” refers to both therapeutic treatment and prophylactic or preventative measures, wherein the aim is to prevent, ameliorate, reduce or slow down (lessen) or improve a condition, disease or disorder.

“Treating” or “treatment” as used herein covers any treatment of, or prevention of a condition in a vertebrate, a mammal, particularly a human.

“Preventing”, “prevention”, “preventative” or “prophylactic” refers to keeping from occurring, or to hinder, defend from, or protect from the occurrence of a condition, disease, disorder, or phenotype, including an abnormality or symptom. A subject in need of prevention may be prone to develop the condition.

The term “ameliorate” or “amelioration” refers to a decrease, reduction or elimination of a condition, disease, disorder, or phenotype, including an abnormality or symptom. A subject in need of treatment may already have the condition, or may be prone to have the condition or may be in whom the condition is to be prevented.

The term “maintain” as used herein refers to sustaining a condition at pre-treatment levels.

The RNAses or growth factors of the second aspect may be provided as a pharmaceutical, veterinary or nutraceutical composition or as a food.

A pharmaceutical composition is one which is suitable for administration to humans. A veterinary composition is one that is suitable for administration to animals. Generally such compositions will contain purified RNAses or growth factors or at the very least all components of the composition will be verifiable.

The pharmaceutical or veterinary composition may comprise one or more carriers and optionally other therapeutic agents. Each carrier, diluent, adjuvant and/or excipient may be “acceptable”.

By “acceptable” is meant a material which is not biologically or otherwise undesirable, i.e., the material may be administered to an individual along with the selected active agent without causing any undesirable biological effects or interacting in a deleterious manner with any of the other components of the pharmaceutical or veterinary composition in which it is contained. Similarly, a “acceptable” salt or ester of a novel compound as provided herein is a salt or ester which is not biologically or otherwise undesirable.

As used herein, a “carrier” is an acceptable solvent, suspending agent or vehicle for delivering the agent to the subject. The carrier may be liquid or solid and is selected with the planned manner of administration in mind. Each carrier must be “acceptable” in the sense of being not biologically or otherwise undesirable i.e. the carrier may be administered to a subject along with the agent without causing any or a substantial adverse reaction.

The pharmaceutical or veterinary composition may be administered orally, topically, or parenterally in formulations containing conventional non-toxic acceptable carriers, adjuvants, and vehicles.

The term parenteral as used herein includes intravenous, intraarterial, intraperitoneal, intramuscular, subcutaneous, subconjunctival, intracavity, transdermal and subcutaneous injection, aerosol for administration to lungs or nasal cavity or administration by infusion by, for example, osmotic pump.

The pharmaceutical or veterinary composition may be administered orally as tablets, aqueous or oily suspensions, lozenges, troches, powders, granules, emulsions, capsules, syrups or elixirs. The composition for oral use may contain one or more agents selected from the group of sweetening agents, flavouring agents, colouring agents and preserving agents in order to produce pharmaceutically elegant and palatable preparations. Suitable sweeteners include sucrose, lactose, glucose, aspartame or saccharin. Suitable disintegrating agents include corn starch, methylcellulose, polyvinylpyrrolidone, xanthan gum, bentonite, alginic acid or agar. Suitable flavouring agents include peppermint oil, oil of wintergreen, cherry, orange or raspberry flavouring. Suitable preservatives include sodium benzoate, vitamin E, alphatocopherol, ascorbic acid, methyl paraben, propyl paraben or sodium bisulphite. Suitable lubricants include magnesium stearate, stearic acid, sodium oleate, sodium chloride or talc. Suitable time delay agents include glyceryl monostearate or glyceryl distearate. The tablets may contain the agent in admixture with non-toxic pharmaceutically acceptable excipients which are suitable for the manufacture of tablets.

These excipients may be, for example, (1) inert diluents, such as calcium carbonate, lactose, calcium phosphate or sodium phosphate; (2) granulating and disintegrating agents, such as corn starch or alginic acid; (3) binding agents, such as starch, gelatin or acacia; and (4) lubricating agents, such as magnesium stearate, stearic acid or talc. These tablets may be uncoated or coated by known techniques to delay disintegration and absorption in the gastrointestinal tract and thereby provide a sustained action over a longer period. For example, a time delay material such as glyceryl monostearate or glyceryl distearate may be employed.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's intravenous vehicles include fluid and nutrient replenishers, electrolyte replenishers (such as those based on Ringer's dextrose), and the like. Preservatives and other additives may also be present such as, for example, anti-microbials, anti-oxidants, chelating agents, growth factors and inert gases and the like.

The pharmaceutical or veterinary composition may also contain other active compounds providing supplemental, additional, or enhanced therapeutic functions. The pharmaceutical or veterinary composition may also be included in a container, pack, or dispenser together with instructions for administration.

The pharmaceutical or veterinary composition can be administered in one dose, or at intervals such as once daily, once weekly, and once monthly.

Dosage schedules can be adjusted depending on the half life of the active agent, or the severity of the subject's condition.

Generally, the pharmaceutical or veterinary composition is administered as a bolus dose, to maximize the circulating levels of active agent for the greatest length of time after the dose. Continuous infusion may also be used after the bolus dose.

The RNAses or growth factors of the second aspect may be provided in a nutraceutical composition or food.

The term “nutraceutical” as used herein refers to an edible product isolated or purified from food, in this case from a milk product, which is demonstrated to have a physiological benefit or to provide protection or attenuation of an acute or chronic disease or injury when orally administered. The nutraceutical may thus be presented in the form of a dietary preparation or supplement, either alone or admixed with edible foods or drinks.

The nutraceutical composition may be in the form of a soluble powder, a liquid or a ready-to-drink formulation. Alternatively, the nutritional composition may be in solid form as a food; for example in the form of a ready-to-eat bar or breakfast cereal. Various flavours, fibres, sweeteners, and other additives may also be present.

The nutraceutical preferably has acceptable sensory properties (such as acceptable smell, taste and palatability), and may further comprise vitamins and/or minerals selected from at least one of vitamins A, B1, B2, B3, B5, B6, B11, B12, biotin, C, D, E, H and K and calcium, magnesium, potassium, zinc and iron.

The nutraceutical composition may be produced as is conventional; for example, the composition may be prepared by blending together the protein and other additives. If used, an emulsifier may be included in the blend. Additional vitamins and minerals may be added at this point but are usually added later to avoid thermal degradation.

If it is desired to produce a powdered nutraceutical composition, the protein may be admixed with additional components in powdered form. The powder should have a moisture content of less than about 5% by weight. Water, preferably water which has been subjected to reverse osmosis, may then be mixed in to form a liquid mixture.

If the nutraceutical composition is to be provided in a ready to consume liquid form, it may be heated in order to reduce the bacterial load. If it is desired to produce a liquid nutraceutical composition, the liquid mixture is preferably aseptically filled into suitable containers. Aseptic filling of the containers may be carried out using techniques commonly available in the art. Suitable apparatus for carrying out aseptic filling of this nature is commercially available.

Preferably the nutraceutical composition also comprises one or more pharmaceutically acceptable carriers, diluents or excipients. Nutraceutical compositions may comprise buffers such as neutral buffered saline, phosphate buffered saline and the like; carbohydrates such as glucose, mannose, sucrose or dextrans; mannitol; proteins; polypeptides or amino acids such as glycine; antioxidants; chelating agents such as EDTA; adjuvants and preservatives.

The nutraceutical may be an infant formula, particularly a humanised milk formula for administration to infants.

When provided as a food the RNAses or growth factors of the second aspect can take the form of a food supplement, a nutritional formulation, a sports nutrition supplement or an infant formula. In one embodiment the food is animal feed.

Throughout this specification, unless the context requires otherwise, the word “comprise”, or variations such as “comprises” or “comprising”, will be understood to imply the inclusion of a stated element or integer or group of elements or integers but not the exclusion of any other element or integer or group of elements or integers.

It must also be noted that, as used in the subject specification, the singular forms “a”, “an” and “the” include plural aspects unless the context clearly dictates otherwise.

It will be apparent to the person skilled in the art that while the invention has been described in some detail for the purposes of clarity and understanding, various modifications and alterations to the embodiments and methods described herein may be made without departing from the scope of the inventive concept disclosed in this specification.

### EXAMPLES

The invention is now further described in detail by reference to the following examples. The examples are provided for purposes of illustration only, and are not intended to be limiting unless otherwise specified. Thus, the invention encompasses any and all variations which become evident as a result of the teaching provided herein.

### Example 1

### Ultrafiltration of Mixtures of Cationic Milk Proteins to Isolate Growth Factors

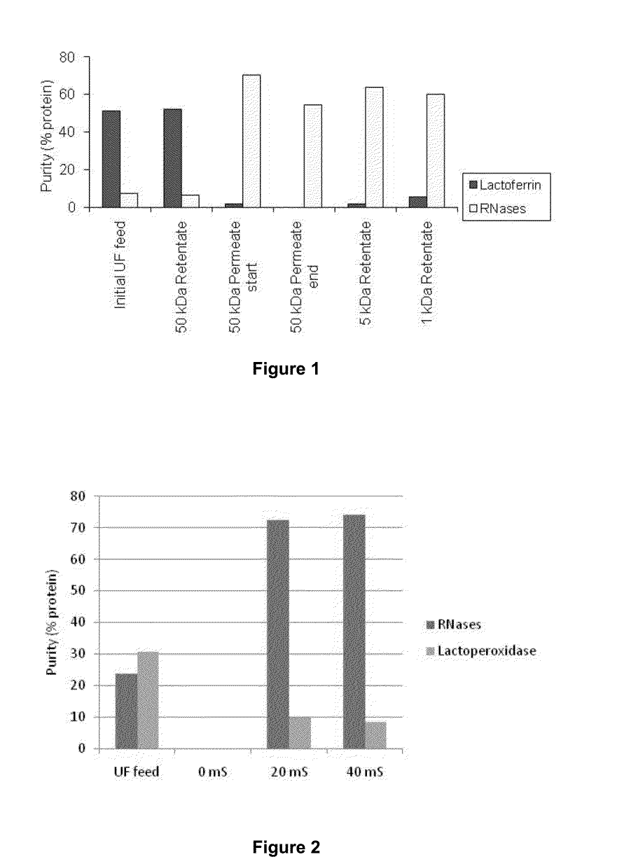

A milk fraction containing a mixture of cationic milk proteins including lactoferrin, lactoperoxidase, RNAses, immunoglobulins and growth factors was subjected to ultrafiltration with a six inch, 50 kDa membrane. The ultrafiltration plant was stabilised so that the baseline pressure was 3.2-3.4 Bar and the transmembrane pressure 1.2-1.4 Bar. The feed contained 10 mg/mL protein, sodium chloride equivalent to 100 mS and water. Feed, 50 kDa retentate, 50 kDa permeate at the start and end of the concentration were sampled. The 50 kDa permeate was pooled and concentrated and desalted by either 5 kDa ultrafiltration membranes (Sartorius Vivacell) or 1 kDa nanofiltration membrane (2.5″ Koch membranes). The 1 kDa retentate is enriched in RNAses (FIG. 1) and growth factors including IGF-1, TGF-β2 and others. Purity was assessed by cation exchange HPLC.

### Example 2

### Ultrafiltration of Mixtures of Cationic Milk Proteins to Isolate Growth Factors

A milk fraction containing a mixture of cationic milk proteins including lactoferrin, lactoperoxidase, RNAses, immunoglobulins and growth factors was subjected to ultrafiltration using a six inch, 50 kDa membrane. The ultrafiltration plant was stabilised so that the baseline pressure was 3.2-3.4 Bar and the transmembrane pressure 1.2-1.4 Bar. The feed contained 10 mg/mL protein, sodium chloride equivalent to 100 mS and water. The ultrafiltration process was tested at 0, 20 and 40 mS. The permeate was collected and analysed by cation exchange HPLC. It was found that at a conductivity of 20 mS or greater, growth factors (RNAses used as an example protein) crossed the membrane, but large molecular weight proteins (lactoperoxidase used as an example protein) did not (FIG. 2). It would be expected that other growth factors, including (although not being limited to this list) IGF-1, IGF-2, PDGF, FGF-basic, EGF, FGF-acidic, VEGF would also be enriched relative to high molecular weight proteins such as lactoferrin and lactoperoxidase.

### Example 3

### Ultrafiltration of Lactoferrin to Remove Small Molecules and Increase Lactoferrin Purity

An ultrafiltration plant was fitted with a single 6 inch 50 kDa membranes (Synder) and then stabilised so that the baseline pressure was 3.2-3.4 Bar and the transmembrane pressure 1.2-1.4 Bar. Lactoferrin solution (2 mg/mL) was split into six lots of 2 L. In six experiments that were identical, except for the conductivity (0, 20, 40 or 60 mS), 2 L lactoferrin solution was added to the UF feed tank, topped to 170 L with water and adjusted to the nominated conductivity by adding sodium chloride solution. Retentate was recycled to the feed tank and permeate was collected. The permeate removed was replaced with diafiltration solution of the same conductivity. Purity was assessed by cation exchange HPLC.

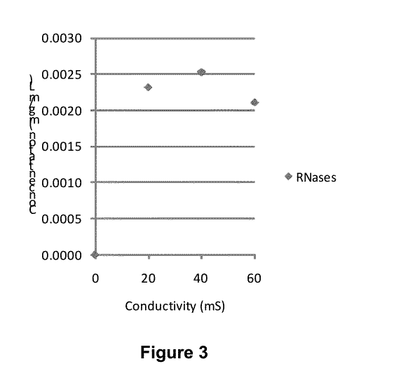

Lactoferrin purity was increased from 90.9% (0 mS) to 93.5% (60 mS), which represents a 2.5% increase in purity. The increase in lactoferrin purity was achieved by the selective removal of growth factors (RNAses used as an example, FIG. 3).

### Example 4

### Ultrafiltration Membranes

Justification of MWCO:

−30 kDa membranes successfully retain the harvested lactoferrin (protein transmission through the membrane is 0.6% of the protein present), which increases purity by allowing key contaminants, especially RNases) to pass through the membranes.

sodium chloride (58 Da) is not retained by the 30 kDa membranes, whereas lactoferrin (80 kDa) is and for this reason lactoferrin eluting from the column at a low concentration (0.1% protein) can be concentrated to 3% protein, without a matching increase in the salt concentration or loss of protein.

### UF5

| | |
|---|---|
| Type: Spiral wound polyether sulphone (PES) | MWCO: 5 kDa |
| Brand: Synder | Model: MT2B-6338 |

The ultrafiltration plant known as UF5 was used to:
1. concentrate non-lactoferrin proteins eluted from the cation exchange column, and
2. recycle the 2.5% salt back to the 2.5% salt tank for reuse in the chromatographic process.
Justification of process within the broader lactoferrin manufacturing process:

sodium chloride (2,000 kg per batch) is a significant cost ($1,200 per batch) in the lactoferrin manufacture process and the ability to recycle sodium chloride dramatically reduces the cost of production.

environmental damage is reduced by recycling sodium chloride. After tertiary treatment at Leongatha to remove organic solids, sodium chloride-containing effluent is disposed of by means of an ocean outfall and all steps must be taken to reduce the amount of waste generated. The salt recycling process reduces the amount of salt required by 80%, meaning that the amount of sodium chloride released into the environment is reduced by 8,000 kg per batch (one-third due to UF5).

### Justification of Membrane Type:

spiral membranes are a comparatively cheap way of obtaining a large area of membrane, which allows high fluxes in a plant with a small foot print.

PES is an inherently hydrophilic membrane that wets out quickly and completely resulting in fast filtration with superior flow rates and high throughputs. PES membrane is also extremely low protein binding minimizing the likelihood of target protein binding, which means high yields, stable transmembrane fluxes and consistent apparent membrane porosities.

### Justification of MWCO:

5 kDa membranes retain the non-lactoferrin impurities, which prevents them from returning to the chromatographic process and contaminating the lactoferrin during subsequent lactoferrin elutions. A smaller membrane is required because the proteins mixture contains many smaller proteins, many of which are growth factors.

sodium chloride (58 Da) is not retained by the 5 kDa membranes, whereas lactoperoxidase (80 kDa), immunoglobulins (150 to 420 kDa) and growth factors (5 to 17 kDa) are and for this reason proteins eluting from the column at a low concentration (0.1% protein) can be concentrated to 3% protein, without a matching increase in the salt concentration or loss of protein.

### UF7

| | |
|---|---|
| Type: Spiral wound polyvinylidene fluoride (PVDF) | MWCO: 50 kDa |
| Brand: Synder | Model: BN4B-6338 |

### The Ultrafiltration Plant Known as UF7 was Used to:

1. increase the lactoferrin purity by reducing the non-lactoferrin proteins; and
2. increase the protein to >95% solids by removing sodium chloride and residual lactose.
Justification of Process within the Broader Lactoferrin Manufacturing Process:

the lactoferrin intended for the manufacture of Ferritin OB has a higher lactoferrin purity than standard lactoferrin. The higher purity is obtained by substituting 50 kDa membranes for the 5 kDa membranes historically used.

### Justification of Membrane Type:

spiral membranes are a comparatively cheap way of obtaining a large area of membrane, which allows high fluxes and relatively short process times in a plant with a small foot print.

hydrophilic polyvinylidene fluoride (PVDF) membranes have high transmembrane fluxes and low affinity for proteins. For these reasons, fluxes remain high for the duration of the process and yields remain high because little protein fouls the membrane pours.

### Justification of MWCO:

50 kDa membranes are used in preference to smaller membranes (historically 5 kDa membranes) because they allow improved transmission of non-lactoferrin proteins (RNases, growth factors), while retaining lactoferrin. The increased transmission of non-lactoferrin protein results in a higher lactoferrin purity (average increase in purity 1.8% protein, P<0.001). Further details can be obtained from ‘Increasing Lactoferrin Purity by Diafiltration with Salt Solution in an Ultrafiltration Plant Fitted with 50 kDa Membranes’ (JR0010).

sodium chloride (58 Da) is not retained by the 50 kDa membranes, whereas lactoferrin (80 kDa) is and for this reason lactoferrin eluting from the column at a low concentration (3% protein) can be concentrated to >20% protein, without a matching increase in the salt concentration or loss of protein.

increases total solids by removing water and therefore maximises freeze-dryer solids throughput.

1. A process for purifying RNAses and growth factors from milk or lactoferrin, the process comprising subjecting the milk or lactoferrin to filtration to separate it into a retentate fraction comprising lactoferrin and a permeate fraction comprising growth factors and/or RNAses, wherein prior to and/or during filtration the milk or lactoferrin is subjected to salt treatment such that growth factors and/or RNAses flow into the permeate.

2. A process for purifying RNAses and growth factors from milk or lactoferrin, the process comprising subjecting the milk or lactoferrin to filtration to separate it into a retentate fraction comprising lactoferrin and a permeate fraction comprising growth factors and/or RNAses, wherein prior to and/or during filtration the milk or lactoferrin is subjected to salt treatment capable of disaggregating or disassociating any mass of RNAses or growth factors such that growth factors and/or RNAses flow into the permeate.

3. A process for purifying RNAses and growth factors from milk or lactoferrin, the process comprising subjecting the milk or lactoferrin to membrane filtration to separate it into a retentate fraction comprising lactoferrin and a permeate fraction comprising growth factors and/or RNAses, wherein prior to and/or during filtration the milk or lactoferrin is subjected to salt treatment capable of separating any RNAses or growth factors from the membrane such that growth factors and/or RNAses flow into the permeate.

4. RNAses and growth factors obtained from the process of any one of claims 1 to 3.

5. Use of the RNAses and growth factors of claim 4 in for the treatment of diseases caused by viruses, bacteria, or fungi and their toxins, to target pathogens which cause infections of human mucosal surfaces, to promote angiogenesis, for treating a disorder characterised by elevated myostatin, for treating disorders where the interaction between follistatin and angiogenin can be used to improve function in tissues, for promoting muscle growth, for improving recovery of muscle from injury or use, for improving muscle strength, for improving exercise tolerance, for increasing the proportion of muscle, for decreasing fat, for decreasing an individual's fat to muscle ratio, for treating neurological diseases or disorders, for treating spinal injuries or diseases, for treating bone diseases or disorders, for treating diseases involving glucose homeostasis, for wound healing, or for providing neuroprotection, nervous system functional support, managing metabolic diseases and/or increasing the bone density of an individual, for treating inflammation, to treat cancer, to treat cancer cachexia, and to treat periodontitis and in all other uses of growth factors and RNAses.
